(19) United States
(12) Patent Application Publication
(10) Pub. No.: US 2006/0134730 A1
(43) Pub. Date: Jun. 22, 2006

(54) Culture medium for detecting micro-organisms

(76) Inventor: Alain Rambach, Paris (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/355,004

(22) Filed: Feb. 16, 2006

Related U.S. Application Data
(63) Continuation of application No. 10/335,895, filed on Jan. 3, 2003.

(30) Foreign Application Priority Data
Jan. 4, 2002 (FR) 02/00076

(51) Int. Cl. C12Q 1/04

(52) U.S. Cl. 435/34

(57) ABSTRACT

A culture medium to detect microorganisms containing carrageenan as a gelling factor, the carrageenan is preferably iota carrageenan. The culture may also comprise an agent releasing oxygen, such as nitrate oxide.

### CROSS-REFERENCE TO RELATED APPLICATION

This application is a continuation of U.S. application Ser. No. 10/335,895, filed Jan. 3, 2003, incorporated by reference herein in its entirety and relied upon.

The present invention relates to a new culture medium to detect micro-organisms, containing a carrageenan, as a gelling factor.

The identification of contaminant micro-organisms is very important, in particular in the fields of food industry or the care hospital.

One can carry out this detection by many means and in particular the amplification of the specific nucleic acids of the required contaminants. However, this method requires the follow-up of a process which cannot always be made in routine.

One can also carry out detection by the growth of the micro-organisms on adequate culture media, and the analysis of the obtained colonies. For this, one can complement the culture medium with elements allowing to improve the identification of the contaminants, for example chromogenic agents, releasing chromophores precipitating in the solid medium, after hydrolysis by specific enzymes of the target micro-organisms.

However, the use of such mediums can prove to be problematic in certain cases. Indeed, the already solidified mediums have a lifespan (shelf-life) rather short and must be preserved preferably in the cold. Thus, one prefers to use dehydrated mediums, being presented in the form of powder, with a longer lifespan on the shelf, and one prepares the medium where necessary.

These dehydrated mediums contain agar as a gelling agent, which has the disadvantage of gelling only while cooling after being put in the presence of hot water. Thus, one generally autoclaves the medium, then pours it in Petri dishes and lets it take in mass. This cannot be carried out when one does not have possibility of heating water to prepare the medium.

It is thus advisable to find a means of preparing gelled culture media, even if one does not have an autoclave, or without using hot water, necessary for the dissolution and gelling of agar.

It was observed that one can manufacture a culture medium by using, as a gelling agent, a carrageenan, and that the medium takes in mass in the presence of water, even cold. The culture medium thus prepared allows the growth of the micro-organisms, as on a medium containing agar.

The carrageenans are polysaccharides resulting from the algae Chondrus crispus, and are well-known in food and cosmetic industries. They exist of three kinds, the kappa, iota, and lambda.

Thus, the present invention refers to a dehydrated culture medium containing a sufficient quantity of carrageenan to allow the formation of a gelled medium after addition of water, said carrageenan being any of the three above mentioned carrageenans, preferably the iota carrageenan, or the kappa carrageenan.

One of the ways of using the product is to add the sample to be analyzed directly on the powdered medium and to mix. The medium then gels in masse and the micro-organisms that are present grow directly within the volume created by the gelled medium (globules in the 3-dimensions medium), which can be a problem if anaerobe is too high in the medium. Thus, in order to allow a better growth of the micro-organisms in the culture medium, one can add, if necessary, an agent releasing oxygen, in particular nitrate, in particular an oxide of nitrate.

In order to reduce the risks of degradation of the product, one preferably packages it safe from ambient moisture. Thus, the invention also refers to a kit for preparing a culture medium comprising a medium according to the invention, conditioned in a container hermetic to moisture. Alternatively, the kit according to the invention comprises a medium according to the invention as well as a dessicator element making it possible to avoid the hydration of the dehydrated medium.

Generally, the invention refers to the use of a carrageenan, in particular the iota carrageenan or the kappa carrageenan, as a gelling agent in a gelled culture medium.

The invention also refers to a method of preparation of a gelled culture medium comprising the step of mixing water with a culture medium according to the invention, in particular when water is at a temperature lower than 50° C., even lower than 40° C.

The culture medium according to the invention contains the nutriments necessary to the growth of the target micro-organisms, and/or the inhibition of certain parasitic micro-organisms and is similar to the mediums known by the person skilled in the art, the agar being replaced by the carrageenan. The medium according to the invention can also contain agents for the identification of the micro-organisms, and in particular chromogenic or fluorogenic agents.

As an example, the composition of a culture medium according to the invention can be as follows (for 1 liter):

| | | |
|---|---|---|
| Iota carrageenan | 25 | g |
| Deoxycholate | 1 | g |
| KNO₃ | 1 | g |
| KH₂PO₄ | 0.66 | g |
| Na₂ HPO₄ | 2.3 | g |
| NaCl | 7.5 | g |
| Peptone | 5 | g |
| Yeast extract | 2 | g |
| Magenta-glucoside | 0.05 | g |
| X-glucuronide | 0.05 | g |
| Meth-glucuronide | 0.1 | g |
| Cellobiose | 0.1 | g |

Such a medium is particularly adapted for the detection of coliforms, but it is understood that the addition of others nutriments or growth elements makes it possible to define mediums to detect other organisms.

What is claimed is:

1. A method for detecting microorganisms comprising:
(1) mixing a sample to be tested with a dehydrated culture medium comprising a carrageenan as the sole gelling agent and nutriments necessary to the growth of the target microorganisms;
2. allowing the mixture to gel and the microorganisms to grow therein; and
3. detecting the microorganisms;
said method being conducted without heating.

2. The method according to claim 1, wherein said carrageenan is iota carrageenan.

3. The method according to claim 1, wherein said culture medium further comprises an oxygen-releasing agent.

4. The method according to claim 3, wherein said oxygen-releasing agent is a nitrate.

5. The method according to claim 4, wherein said nitrate is KNO₃.

6. A method for detecting microorganisms comprising:
(1) mixing water which is at a temperature of at least 40° C. but less than 50° C., with a dehydrated culture medium comprising a carrageenan as the sole gelling agent and nutriments necessary to the growth of target microorganisms;
(2) allowing the mixture to gel, adding a sample to be tested and allowing the microorganisms to grow on the gel; and
(3) detecting the microorganisms;
said method being conducted without heating except for the temperature of the water at the start.

7. The method according to claim 6, wherein said carrageenan is iota carrageenan.

8. The method according to claim 6, wherein said culture medium further comprises an oxygen-releasing agent.

9. The method according to claim 8, wherein said oxygen-releasing agent is a nitrate.

10. The method according to claim 9, wherein said nitrate is KNO₃.
